(19) United States
(12) Patent Application Publication
(10) Pub. No.: US 2022/0054575 A1
(43) Pub. Date: Feb. 24, 2022

(54) PROTECTION AGAINST CORONAVIRUS INFECTION BY EXTRACTS AND EXTRACT COMPONENTS

(71) Applicant: Natreon, Inc., New Brunswick, NJ (US)

(72) Inventor: Sanyasi R. Kalidindi, Monroe, NJ (US)

(73) Assignee: Natreon, Inc., New Brunswick, NJ (US)

(21) Appl. No.: 17/405,720

(22) Filed: Aug. 18, 2021

Related U.S. Application Data
(60) Provisional application No. 63/067,667, filed on Aug. 19, 2020.

(51) Int. Cl. A61K 36/81; A61K 35/10; A61K 31/353; A61P 31/14

(52) U.S. Cl. CPC A61K 36/81; A61P 31/14; A61K 31/353; A61K 35/10

(57) ABSTRACT

This disclosure is directed to methods of treating, preventing, and protecting against viral infection including coronavirus infection and symptoms such as in COVID-19 infection. The methods include the administration of a composition comprising an extract and/or extract component, specifically, plant and/or herbo-mineral extracts, such as of Shilajit, Withania somnifera, Asparagus racemosus, Terminalia chebula, Terminaha bellirica, Phyllanthus emblica, Azadirachta indica, and a trivalent chromium complex with extracts of P. emblica and Shilajit; and/or extract components such as 3,8-dihydroxy-6H-benzo[c]chromen-6-one and 3-hydroxy-6H-benzo[c]chromen-6-one.

### CROSS REFERENCE TO RELATED APPLICATIONS

This application claims the benefit of U.S. Provisional Application No. 63/067,667, filed Aug. 19, 2020. The provisional application is incorporated by reference in its entirety herein.

### FIELD OF INVENTION

This invention relates to methods of treating, preventing, and protecting against viral infection including coronavirus infection such as COVID-19 with natural extracts and/or their components. The methods include administering plant and/or herbo-mineral extracts such as extracts of Shilajit, Withania somnifera, Asparagus racemosus, Terminaha chebula, Terminaha bellirica, Phyllanthus emblica, Azadirachta indica, and/or a trivalent chromium complex with extracts of P. emblica and Shilajit; and also administering extract components such as 3,8-dihydroxy-6H-benzo[c]chromen-6-one and 3-hydroxy-6H-benzo[c]chromen-6-one.

### BACKGROUND

COVID-19 is a viral infection caused by severe acute respiratory syndrome coronavirus 2 (SARS-CoV-2). The virus was first known in Wuhan, China in the later part of 2019 and quickly instigated a worldwide pandemic. As of the end of July 2021, over 200 million people worldwide have been infected, with more than 4,000,000 deaths. The virus spreads through air and contact transmission. Some infected people show no symptoms but can spread the infection. For those who show symptoms, it takes up to 14 days to show the symptoms, which primarily include fever, dry cough and difficulty breathing. The COVID-19 virus causes a wide range of pathological manifestations leading to respiratory failure, blood clots, multiple organ damage, heart attack, stroke, and other serious complications. Some infected people recover quickly with no residual problems, while others may take a long time to recover or succumb to the infection. This infection is particularly dangerous to elderly people; however, younger people are also at risk for severe infection. The world economy, especially that of the USA, has been adversely affected because of the lockdown of businesses due to the COVID-19 pandemic. The infection is under control in some countries but still raging in some other countries.

Precautionary measures, such as wearing a mask in public, frequent hand washing with soap, and physical distancing, help control the infection. Treatment options are limited at present. Several anti-viral drugs have been studied for treatment and one of them, remdesivir, seems to hold some promise. Prevention using vaccination is the best option to eradicate the virus. Several vaccines are under development at the current time.

Natural products offer an attractive alternative to drugs for treatment and/or prevention of COVID-19 infection. The present invention relates in part to anti-COVID-19 activity of several natural plant and herbo-mineral extracts.

Shilajit, also known as “Moomiyo,” is found in high altitudes, for instance of the Himalayan Mountains, and is considered one of the “wonder medicines” of Ayurveda, the traditional Indian system of medicine dating back to 3500 B.C.E. Shilajit is physiologically active organic matter, composed of rock humus, rock minerals, and organic substances that have been compressed by layers of rock mixed with marine organisms and microbial metabolites. Shilajit oozes out of the rocks as a black mass in the Himalayas at higher altitudes ranging from 1000 to 5000 meters, as the rocks become warm during summer. Shilajit contains fulvic acids (“FAs”) as its main components, along with dibenzo-α-pyrones (“DBPs”, also known as chromen-6-ones, such as 3,8-dihydroxy-6H-benzo[c]chromen-6-one (3,8-(OH)₂-DBP; DBP A; Urolithin A) and 3-hydroxy-6H-benzo[c]chromen-6-one (3-OH-DBP; DBP B; Urolithin B)) and DBP chromoproteins, humic acid, and more than forty (40) minerals.

Withania somnifera, commonly known as Ashwagandha, has been used in herbal formulations of the Ayurvedic or Indian system of medicine, for instance to help to ward off stress and act as an adaptogen.

Terminaha chebula has been extensively used in Ayurveda, Unani and Homeopathic systems of medicine for improvement of different health conditions. T. chebula may be rich in tannoids and may contain a variety of other constituents. Chemical constituents isolated from T. chebula may vary considerably in type and/or concentration due to a number of factors, e.g., ecological variation, soil variation, and nutrient variation, as well as variations in the process of extraction.

Terminaha bellerica (also belerica, belirica, bellirica) is grown widely throughout India, Sri Lanka, and South East Asia. T. bellerica has been used for centuries in Ayurveda, and may contain several chemical constituents in common with T. chebula.

Phyllanthus emblica, the Indian gooseberry, is also widely used in Indian medicine for the treatment of various diseases.

Azadirachta indica, commonly known as neem, nitree, or Indian lilac, is a tree in the mahogany family Meliaceae. It is one of two species in the genus Azadirachta, and is native to the Indian subcontinent, i.e., India, Nepal, Pakistan, Bangladesh, Sri Lanka, and Maldives. It is typically grown in tropical and semi-tropical regions. Neem trees also grow in islands located in the southern part of Iran. Its fruits and seeds are the source of neem oil.

Asparagus racemosus is a popular plant in the ancient Ayurvedic tradition that is believed to improve vitality, immunity, and vigor. Asparagus racemosus has previously been demonstrated to elicit antitussive, antibacterial, antihepatotoxic, immunomodulatory, and antioxidant effects in both rat and human models. The primary phytochemicals found in Asparagus racemosus include saponins, such as shatavarin VI and shatavarin VII, as well as antioxidants such as asparagamine A, racemosol, and racemofuran.

A unique trivalent chromium complex prepared by complexing Chromium III with two natural products, Phyllanthus emblica and Shilajit, such as Crominex®-3+, is useful in various applications.

### BRIEF DESCRIPTION OF THE DRAWINGS

FIG. 1 is a series of photomicrographs showing phase contrast images of MRC-5 cells pre-treated with PrimaVie® Shilajit and infected with coronavirus 229E.

FIG. 2 is a series of photomicrographs showing reduced cell death and improved cell viability in MRC-5 cells infected with coronavirus 229E and treated with PrimaVie® Shilajit.

FIG. 3 is a bar graph showing that treatment of MRC-5 cells with PrimaVie® Shilajit decreased cell death from coronavirus about 8 fold, compared to cell death levels in non-infected cells. In each group of 2 bars (0, 0.25, 0.5 mg PrimaVie® Shilajit/ml), the left bar refers to non-infected cells, and the right bar refers to cells infected with 229E. As shown in this Figure, PrimaVie® Shilajit treatment significantly reduced and (0.5 mg/ml) appears to have eliminated cell death from coronavirus 229E infection.

FIG. 4 is a bar graph showing in part that treatment of MRC-5 cells with PrimaVie® Shilajit protected the viability of cells exposed to coronavirus. In each group of 2 bars (0, 0.25, 0.5 mg PrimaVie® Shilajit/ml), the left bar refers to non-infected cells, and the right bar refers to cells infected with 229E. As shown in this Figure, PrimaVie® Shilajit treatment significantly increased cell viability in cells infected with coronavirus 229E.

### SUMMARY OF INVENTION

The present invention is directed to a method of treating and/or preventing viral infection in a subject in need of such treatment, comprising the steps of providing a composition comprising an extract and/or extract component, in an embodiment a standardized aqueous extract, of Shilajit (for instance, PrimaVie®), of Withania somnifera (for instance, Sensoril®), of Terminalia chebula (for instance, AyuFlex®), Terminalia bellerica (for instance, Ayuric®), Phyllanthus emblica (for instance, Capros®), Asparagus racemosus (for instance, Vytus-AR®), Azadirachta indica (for instance, PhytoBGS®), and/or a trivalent complex with extracts of Shilajit and P. emblica (Crominex®-3+), and/or in an embodiment said composition comprising an extract component such as 3,8-dihydroxy-6H-benzo[c]chromen-6-one (3,8-(OH)₂-DBP) and/or 3-hydroxy-6H-benzo[c]chromen-6-one (3-OH-DBP); and administering the composition to the subject in an amount effective to treat and/or prevent the viral infection in the subject, for instance by slowing or stopping viral infection in individual cells in the subject and/or slowing or stopping the rate of viral infection in a group of cells, tissue(s), or organ(s) in the subject.

The present invention is also directed to a method of protecting cells from viral infection comprising the steps of providing a composition comprising an extract and/or extract component, in an embodiment a standardized aqueous extract, of Shilajit (for instance, PrimaVie®), of Withania somnifera (for instance, Sensoril®), of Terminalia chebula (for instance, AyuFlex®), Terminalia bellerica (for instance, Ayuric®), Phyllanthus emblica (for instance, Capros®), Asparagus racemosus (for instance, Vytus-AR®), Azadirachta indica (for instance, PhytoBGS®), and/or a trivalent complex with extracts of Shilajit and P. emblica (Crominex®-3+), and/or in an embodiment said composition comprising an extract component such as 3,8-dihydroxy-6H-benzo[c]chromen-6-one and/or 3-hydroxy-6H-benzo[c]chromen-6-one (respectively, 3,8-(OH)₂-DBP and 3-OH-DBP); and administering an effective amount of the composition to provide protection to the cells from coronavirus infection.

In an embodiment, in a method of protecting, treating, or otherwise exposing the cells to an effective amount of an extract, extract component, and/or composition of this invention, administration may be prior to, concomitant with, or after exposure of the cells to a virus such as a coronavirus such as 229E or SARS-CoV-2. Said effective amount protects cells from viral infection for instance by slowing or stopping infection of individual cells with the virus and/or slowing or stopping the rate of viral infection in a group of cells for instance in a subject.

### DETAILED DESCRIPTION

The below definitions and discussion are intended to guide understanding but are not intended to be limiting with regard to other disclosures in this application. References to percentage (%) in compositions of the present invention refers to the % by weight of a given component to the total weight of the composition being discussed, also signified by “w/w”, unless stated otherwise.

“Viral infection” and the like according to this invention refers to a virus entering the body of a subject and/or a cell, typically with the virus proliferating inside the subject's body and/or the cell. Viral infection of a cell typically entails a virus penetrating a cell, making copies of itself for instance via replication of its nucleic acids and assembly of viral components into new viruses, and releasing the new viruses into the body. A viral infection of a cell typically results in the death of the infected cell. In an embodiment, a cell exposed to a composition of the present invention is protected by the extract and/or extract component in the composition, such that the virus is not able to or on average in a group of cells is less likely and/or able to penetrate the cell, make copies of itself, and kill the cell. In an embodiment, a virus according to the present invention is a coronavirus such as 229E or SARS-CoV-2. Viral infection with SARS-CoV-2, as indicated above, is also called COVID-19. In an embodiment, protection of a cell from viral infection is by PrimaVie®, Urolithin A, Urolithin B, and/or Sensoril®.

Viral infection of a subject refers to the presence of a virus in the subject's body, and typically includes viral infection of at least some of (a “group” of) the subject's cells, where the group of cells may be spread throughout the body, may be the same or similar cell type or different cell types, may share a location in the body (such as the throat or the lungs) and/or share a function and/or structural characteristics. A “subject” according to the present invention is an animal that may be infected by a virus; in an embodiment, a coronavirus; in an embodiment, SARS-CoV-2. In an embodiment, a subject of this invention is a mammal such as a bat, cat, dog, horse, or human; in an embodiment, the subject is a human. In an embodiment, a subject of this invention is a domesticated or herd mammal.

A subject may be symptomatic or asymptomatic when infected with a virus. Symptoms of a SARS-CoV-2 coronavirus infection in a human subject (COVID-19) may include muscle ache, body ache, headache, new loss of taste or smell, fever, chills, cough, shortness of breath and/or other difficulty breathing, fatigue, sore throat, congestion, runny nose, nausea, vomiting, diarrhea and/or other symptoms. In an embodiment, a composition comprising an extract and/or extract component of this invention may be administered in an effective amount to treat and/or prevent a viral SARS-CoV-2 infection in a subject including the subject's cells, thereby slowing or stopping infection of the subject's cells and resulting cell damage and death and related damage to the subject's body, and preventing further cellular infections and damage. Such treatment and prevention may lessen or eliminate symptoms and/or prevent symptoms from manifesting in the subject. In an embodiment, symptoms of other viral infections in a subject, for instance other coronavirus infections, are lessened and/or eliminated with the administration of a composition of this invention. In an embodiment, a treatment according to the present invention lessens and/or eliminates symptoms of a viral infection, including SARS-CoV-2 infection, in the subject.

A viral infection may damage a subject's body including cells, tissue(s), organ(s), and/or organ system(s). Treatment and/or prevention of a viral infection according to this invention may include treatment and/or prevention of damage to (and protection of) the subject's cells, tissue(s), and organ(s). In an embodiment, the viral infection is infection with the SARS-CoV-2 virus and the damage treated and/or prevented by a composition of this invention is to cells, tissue(s), or organs such as of a lung, heart, blood vessel, brain, kidney, and/or pancreas. In an embodiment, such treatment and/or prevention provides protection from serious complications in a subject having COVID-19, such as pneumonia, respiratory failure, blood clots, multiple organ damage, organ failure, heart attack, and others.

The risk of illness including serious illness by a viral infection in a subject may be increased in the subject by an underlying condition (e.g., a pre-existing condition and/or morbidity). For instance, the risk of serious illness in a SARS-CoV-2 infection (COVID-19) in a subject may be increased by an underlying respiratory condition, such as asthma or chronic lung disease such as cystic fibrosis or chronic obstructive pulmonary disease (COPD); by an underlying cardiovascular condition such as high blood pressure, heart disease such as heart failure, coronary artery disease, or cardiomyopathy; by other diseases and conditions such as obesity including severe obesity, cancer, chronic kidney disease, liver disease, sickle cell disease, diseases of the brain and/or nervous system, type 1 diabetes, type 2 diabetes, and/or a weakened immune system for instance from medications that suppress the immune system or an immuno-deficiency virus. In an embodiment, administration of a composition according to the present invention decreases the risk of serious illness in a subject having a viral infection, such as a SARS-CoV-2 infection.

“Administering”, “administration”, and the like, according to the present invention, refers to providing a composition comprising an extract and/or extract component of the present invention to a subject so that the extract and/or extract component may reach the subject's cells and tissues and act on them to protect them from infection by a virus. In an embodiment, the virus is a coronavirus. In an embodiment, the virus is 229E or a severe acute respiratory syndrome coronavirus such as SARS-CoV-2. Without being bound by theory, the extract and/or extract component protects the subject's cells and tissues and prevents cell death for instance by preventing the virus from entering a cell, for instance by inhibiting viral attachment and/or penetration, and/or by preventing the virus from replicating and/or assembling within a cell and/or being released from the cell. In an embodiment, the extract and/or extract component may reach and protect the subject's cells and tissues and act there for instance through the bloodstream and/or for respiratory cells and tissues and organs through the respiratory system (e.g. nose, mouth, pharynx (throat), larynx (voice box), trachea (windpipe), bronchi, lungs, alveoli), with for instance mechanical delivery of the extract and/or extract component or for instance delivery via respiration or other means.

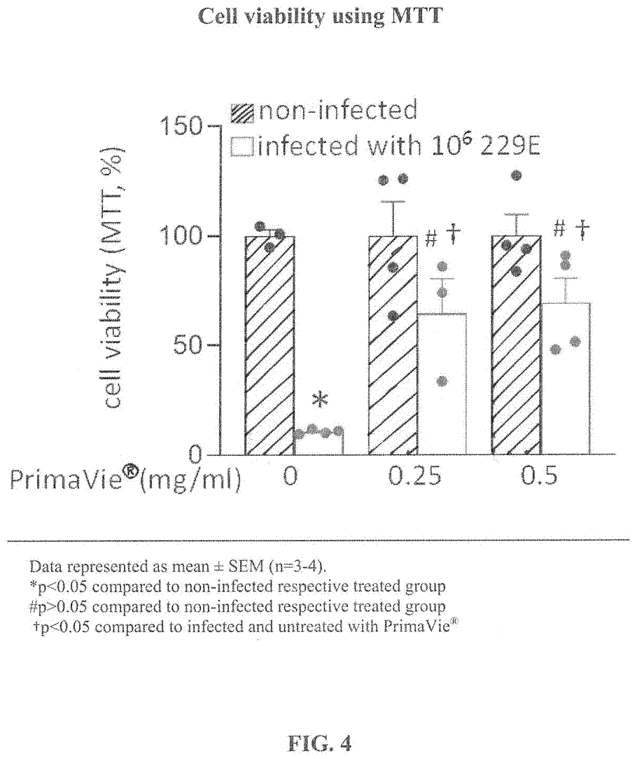

In an embodiment, protection against viral infection by an extract and/or extract component of a composition of this invention maintains cell viability in the presence of the virus. For instance, cells exposed to a virus may become infected and unable to function in a normal manner, and/or may be killed by the virus. The viability of an infected cell, and/or the average viability of a group of cells, may be reduced from for instance 100% viability (healthy functioning cells) to for instance 0%-30% upon infection by a virus such as a coronavirus such as SARS-CoV-2. Cell(s) and/or a group of cells may be protected by an extract and/or extract component of this invention by fully maintaining viability or average viability (e.g. 100%) or by partially maintaining viability or average viability (e.g. 40-90%, such as 40-60%, or 60-80%) as compared with viability before exposure to the virus including viability on average before exposure to the virus. In an embodiment, protecting cell viability or average cell viability according to this invention is exemplified as shown in FIG. 4. Such protection may occur in a subject.

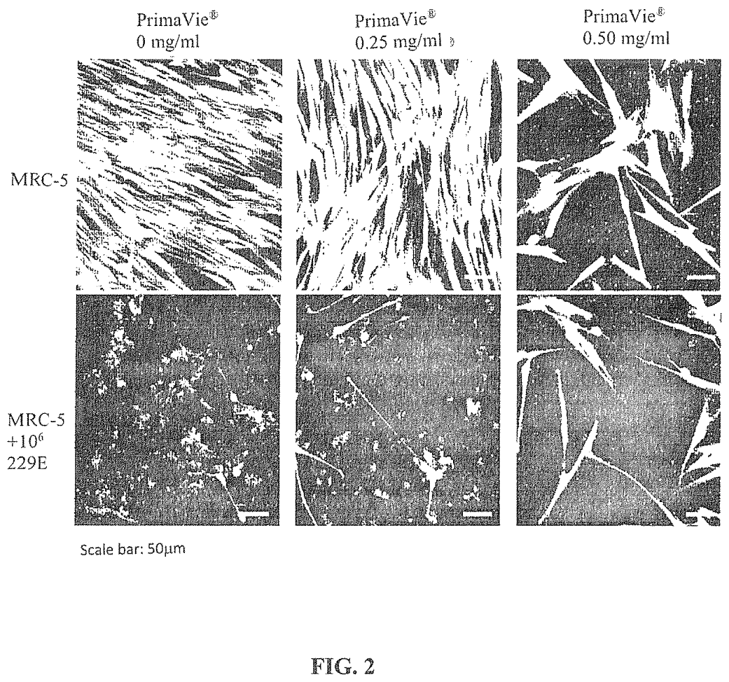

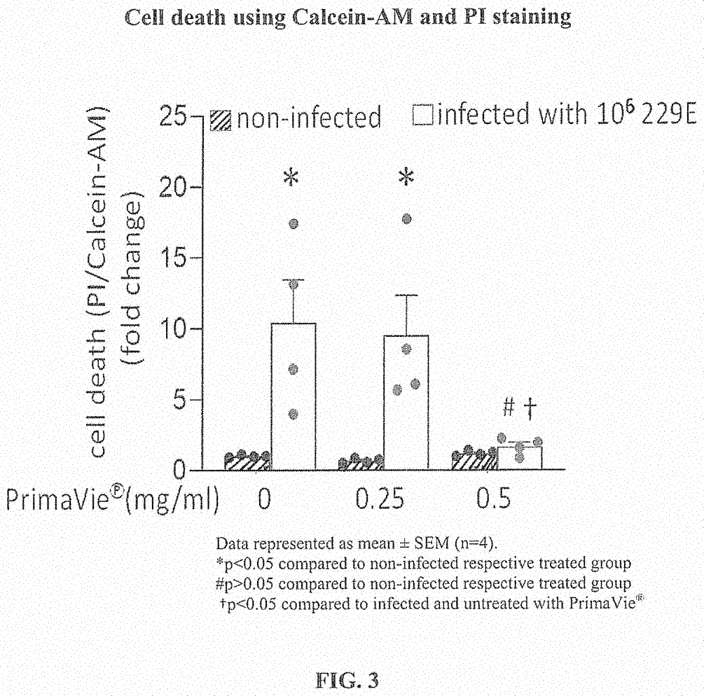

In an embodiment, protection against viral infection by an extract and/or extract component of a composition of this invention reduces cell death in the presence of the virus. For instance, cells exposed to a virus may be killed by viral infection. Protection of cell(s) and/or a group of cells by an extract and/or extract component of this invention may be achieved by preventing an increase in cell death by the viral infection. For instance, a viral infection may increase cell death in a group of cells 2, 3, 4, 5-fold or more. In an embodiment, preventing an increase in cell death includes maintaining cell death at a level normal for a given cell type, for instance as shown in part in FIGS. 2 and 3. Such protection may occur in a subject.

Administration may be by the subject or by another. Administration may be oral, for instance in the form of a dietary supplement, and/or in a solid dosage form, such as in a discrete dose unit, such as a capsule. Administration may also be through parenteral routes such as intravenous, intramuscular, transdermal, subcutaneous, and other physiologically acceptable routes. Administration may be to and via the respiratory system, for instance via topical application, a nasal or sinus rinse, or inhalation through the nose or the mouth, for instance in the form of an aerosol. Administration may be via a rinse or toothpaste in the subject's mouth, an eye drop to protect the subject's eye, an ear drop, or a topical formulation for the skin such as a lotion, ointment, aqueous solution, and so forth. Administration according to the present invention comprises treatment or application of a composition comprising an extract and/or extract component of this invention for instance as shown in the below Examples or as described elsewhere herein. In an embodiment, administration of an effective amount of a composition of this invention includes an amount of about 1 mg to 2000 mg extract daily to a human subject, including about 100-1000 mg extract component daily. In an embodiment, administration of an effective amount of extract and/or extract component to a human subject may be about 100-1000 mg daily, for instance about 50-500 mg twice daily. In an embodiment, a daily dose of an extract or extract component of this invention may be about 1-10 mg/kg; for non-human animals, the effective amount may be based on effective amounts for humans, with an adjustment for individual animal weight. In an embodiment, a composition is administered daily for 1-7 days, preferably consecutive days, or more, for instance for 1-14 days, 1-21 days, 1 month, 2 months, or more, preferably on consecutive days throughout administration, or administered chronically (more than 2 months), or for another period of time according to the present invention.

A “composition” of the present invention comprises an extract from a naturally-occurring source, and/or an extract component. In an embodiment, a composition of this invention comprises, consists essentially of, or consists of any one of the extracts or components of this invention, or any combination of said extracts or components. In an embodiment, a plant (or “herbo-”) “extract” of the present invention may be prepared by disrupting a plant (or its desirable parts) from its natural state (preferably cut into small pieces or crushed/lacerated/etc.) and contacting such with a solvent such as water or buffer (aqueous solution for instance with a specific salt, pH, and/or chemical), to form an aqueous extract; or for instance contacting the plant with a solvent such as ethanol, to form an ethanol extract. In an embodiment, the disrupted plant is partially or fully covered with the solvent. Combinations of solvent applied together or in succession may be used to prepare an extract in an embodiment of this invention. Compounds from the plant move into the water or aqueous solution or alcohol or other solvent to form the extract, plant parts are typically removed and insoluble substances may be filtered out, and if the extract is standardized (e.g. “standardized aqueous extract” or “standardized hydroethanolic extract”), then amounts of specific components of the extract and various characteristics of the extract may be measured and identified to confirm the extract meets the requirements set for this type of extract. In an embodiment, an extract prepared with water and ethanol may be named a hydroethanolic extract. Often, extract standards are summarized in a Report or Certificate of Analysis. Similarly, an herbo-mineral substance like Shilajit may be prepared by combining aqueous solution (e.g. water) or another solvent with naturally-occurring Shilajit. An “extract component” of this invention may be purified from an extract of this invention or otherwise acquired from a natural source, or for instance synthesized. In an embodiment, a composition of this invention comprises a plant extract and/or an herbo-mineral extract of Shilajit, Withania somnifera, Asparagus racemosus, Terminalia chebula, Terminaha bellirica, Phyllanthus emblica, Azadirachta indica, and/or a trivalent chromium complex with extracts of P. emblica and Shilajit; and/or the composition comprises extract components such as 3,8-dihydroxy-6H-benzo[c]chromen-6-one and/or 3-hydroxy-6H-benzo[c]chromen-6-one (respectively, 3,8-(OH)₂-DBP and 3-OH-DBP). In an embodiment, a composition of this invention comprises an extract component that is present in a form more concentrated than its natural source, for instance in an embodiment, in a concentration of about 2×-100× the amount in a naturally occurring source, including for instance 5×-10×, 10×-20×, 20×-50×, and the like, including any number including or within said ranges. In an embodiment, the component is about 25%-100% pure, or in an embodiment, for instance for an isolated component, at least about 90%, 95%, or 99% pure.

In an embodiment, an extract of this invention is a standardized aqueous extract such as PrimaVie® (Shilajit), AyuFlex®, Ayuric®, Capros®, Sensoril®, Vytus-AR®, and/or PhytoBGS®, for instance as summarized and described in Table 1. A composition of the present invention may comprise, consist essentially of, or consist of, an extract of this invention. Specific components of extracts of this invention are identified as present in a minimum or maximum amount or within a specified range, so as to render the extract consistent at least with regard to those components from one batch to the next (“standardized”, as also discussed above).

In an embodiment, a composition of this invention includes an herbo-mineral trivalent chromium complex of standardized aqueous abstracts such as Crominex®-3+, as listed in Table 1. In an embodiment, a composition of this invention includes extract components 3,8-dihydroxy-6H-benzo[c]chromen-6-one (Urolithin A; 3,8-(OH)₂-DBP) and/or 3-hydroxy-6H-benzo[c]chromen-6-one (Urolithin B; 3-OH-DBP), as listed in Table 1. In an embodiment, a composition of this invention comprises, consists essentially of, or consists of a trivalent chromium complex such as Crominex®-3+, 8-dihydroxy-6H-benzo[c]chromen-6-one, and/or 3-hydroxy-6H-benzo[c]chromen-6-one.

TABLE 1
Exemplary extracts and extract components of the present invention

| Number | Preparation type | Source | Example |
|---|---|---|---|
| 1 | Standardized aqueous extract | Shilajit | PrimaVie ® |
| 2 | Standardized aqueous extract | Withania somnifera | Sensoril ® |
| | | roots + leaves | |
| 3 | Standardized aqueous extract | Asparagus racemosus | VytusAR ® |
| | | roots | |
| 4 | Standardized aqueous extract | Terminalia chebula | AyuFlex ® |
| | | fruits | |
| 5 | Standardized aqueous extract | Terminalia bellerica | Ayuric ® |
| | | fruits | |
| 6 | Standardized aqueous extract | Phyllanthus emblica | Capros ® |
| | | fruits | |
| 7 | Standardized aqueous extract | Azadirachta indica | PhytoBGS ® |
| | | leaves + twigs | |
| 8 | Trivalent chromium complex | P. emblica fruits and | Crominex-3+ ® |
| | with standardized aqueous | Shilajit | |
| | extracts | | |
| 9 | Extract component | Natural or Synthetic | 3,8-dihydroxy-6H- |
| | | | benzo[c]chromen-6- |
| | | | one (Urolithin A; 3,8- |
| | | | (OH)₂-DBP) |
| 10 | Extract component | Natural or Synthetic | 3-hydroxy-6H- |
| | | | benzo[c] chromen-6- |
| | | | one (Urolithin B; 3- |
| | | | OH-DBP) |

The compositions described below are intended as representative but not limiting, and may differ depending on the analytical method used. Natural products often contain multiple bioactives for which reference standards may not be readily available in the market.

Shilajit (e.g. PrimaVie®)

In an embodiment, PrimaVie® Shilajit of this invention is a standardized aqueous extract of Shilajit, an herbo-mineral exudate from the Himalayan, Altai and other mountains, brown color powder and soluble in water with earthy/mildly bitter taste. In an embodiment, a Shilajit composition of this invention contains not less than 50% w/w of fulvic acids with DBP core nucleus and not less than 10.3% w/w of free dibenzo-α-pyrones and dibenzo-α-pyrone chromoproteins (DCPs) combined. Dibenzo-α-pyrones are also known as Urolithins, for example Urolithin A and Urolithin B. In an embodiment, PrimaVie® as a composition of this invention is a standardized aqueous extract containing 56-63% fulvic acids with a dibenzo pyrone core nucleus and 12-18% of free dibenzo-α-pyrones and dibenzo-α-pyrones conjugated with chromoproteins combined. PrimaVie® Shilajit used in the below Examples tested at 14.05% w/w DBPs plus DCPs and 58.72% w/w fulvic acids with DBP core nucleus. In addition, the PrimaVie® Shilajit used in the below Examples had about 2.73% moisture content (standard requirement: not more than 6% w/w), pH 7.86 in 2% aqueous solution (standard requirement: greater than pH 5), F4/E6 at 465/665 nm calculated at 7.12 nm (standard requirement: 7-9.5), water soluble extractive value 82.25% w/w by gravimetry (standard requirement: not less than 80% w/w), particle sized with 100% of particles passing through mesh #40 and 97.99% passing through mesh #80 (standard requirement:95% and 85% respectively). The bulk density of the PrimaVie® Shilajit was reported as 0.4 g/cc.

In an embodiment, Sensoril® of this invention is a standardized aqueous extract of the roots and leaves of Withania somnifera plant, brown color powder and soluble in water with bitter taste. It contains not less than 10% w/w of withanolide glycosides, less than 0.5% w/w of withanolide aglycones (as Withaferin-A) and not less than 32% w/w oligosaccharides as bioactives. Withanolides, such as withastromolide, withanolide A, withanolide B, 27-hydroxy withanone, withanone, etc. may also be present in this extract. In an embodiment, Sensoril® as a composition of this invention contains about 10-12% w/w of withanolide glycosides, about 0-0.5% w/w of withanolide aglycones (as Withaferin-A), and 35-40% w/w oligosaccharides, as used in the below Examples; in addition, the Sensoril used in the below Examples had a particle size with 99.50% passing through 40 mesh size and 98.00% passing through 80 mesh size (standard requirement: greater than 95% or 85% respectively); a bulk density of 0.61, with not more than 8% w/w insoluble (inorganic) material, not more than 6% w/w moisture content (measured at about 4.2% w/w), and not less than 80% extractive value.

In an embodiment of the present invention, an extract of Withania somnifera is made by using ethanol extraction followed by water extraction. This extract is referred to as Sensoril®-AWE (“Alcohol-Water-Extract”). Other alcohols, such as methanol, isopropyl alcohol, etc. may also be used as extraction solvents instead of ethanol. Such a hydro-alcoholic Sensoril®-AWE extract of this invention contains not less than 10% w/w withanolide glycosides, not less than 2%, preferably not less than 3.0% w/w withanolide aglycones (as Withaferin-A) and not less than 20% w/w oligosaccharides as bioactives. Withanolides, such as withastromolide, withanolide A, withanolide B, 27-hydroxy withanone, withanone, etc. may also be present in this extract. In an embodiment, a composition of this invention is a hydroethanolic Withania somnifera (Sensoril®-AWE) extract containing 12-20% w/w withanolide glycosides (including as mentioned elsewhere in this application ranges therein, such as 12% w/w, 13% w/w, and so on), 2-8% w/w withanolide aglycones (as Withaferin-A), and 24-35% w/w oligosaccharides.

Asparagus racemosus (e.g. Vytus-AR®)

In an embodiment, a composition according to the present invention comprises a standardized aqueous extract of Asparagus racemosus. In an embodiment, a standardized, powdered, aqueous extract of Asparagus racemosus includes one or more of the following characteristics: Appearance: a free flowing powder, yellowish-brown in color, with a slightly bitter taste, and a water-soluble extractive value of about 90-93%, for instance in the Example below about 90.4% (w/w). In an embodiment, by assay, total saponins (by gravimetry) of the standardized powdered aqueous extract of Asparagus racemosus are at least 38% (w/w) of the extract, for instance about 42% (w/w) in the Asparagus racemosus of the Example below; total amino acids (w.r.t. Arginine and Methionine) of about 4-7% (w/w), for instance 5.79% for the Example below; moisture content (by Karl-Fischer titration) not more than 6%, for instance 3.74% (w/w) for the Example below; sulfated ash content not more than 6% (w/w), for instance 4.9% (w/w) for the Example below. In an embodiment, regarding the particle size of a powdered Asparagus racemosus extract, 100% may be able to pass through mesh #40 and 97-99% through mesh #80 (w/w). In an embodiment, the bulk density of the extract may be about 0.2-0.6 grams/cubic cm, for instance 0.38 gm/cc for the Asparagus racemosus extract used in the below Example. In an embodiment, the tapped density may be of about 0.4-0.5 grams/cubic cm. In an embodiment, Asparagus racemosus is stored in sealed containers at 15° C. to 25° C. In an embodiment, said storage avoids light. In an embodiment, the powdered Asparagus racemosus extract described above is stable for at least 3 years.

Terminaha chebula (e.g. AyuFlex®)

According to the present invention, AyuFlex® is a standardized aqueous extract of the fruits of the Terminaha chebula plant, off white to brown color powder and soluble in water with astringent taste. It contains not less than 39% w/w low molecular weight hydrolysable tannins as bioactives with not less than 27% w/w chebulinic acid and chebulagic acid combined and not less than 12% w/w of other unindentified low molecular weight hydrolysable tannins. Ellagic acid and gallic acid are also present in the extract, for which the analytical results may be reported without any specification, but specifications for these bioactives may also be identified, for instance in an embodiment, each may be present in amounts of less than 10% of the extract or composition. In an embodiment, an aqueous extract of this invention, Ayuflex®, contains about 65-70% w/w low molecular weight hydrolysable tannins including about 45-50% w/w chebulinic acid and chebulagic acid combined. In an embodiment, said aqueous extract may have a measured amount of 8-9% each of gallic acid and ellagic acid. In the below Example, the AyuFlex® used included 68.22% w/w low molecular weight hydrolysable tannins, including 32.79% w/w chebulinic acid+chebulagic acid, and 35.43% w/w other low molecular weight hydrolysable tannins; 4.90% w/w gallic acid; 2.57% w/w ellagic acid; a moisture content 3.95% w/w (standard requirement is not more than 6%), and a water-soluble extractive value of 98.71% w/w (standard requirement is not less than 80%). In an embodiment, a composition of this invention may be a preparation of Terminaha chebula fruit, dried and powdered and in an embodiment, standardized for pharmaceutical or nutraceutical usage. In an embodiment, an extract of this invention may be prepared from such a fruit preparation.

Terminaha bellerica (e.g. Ayuric®)

According to the present invention, Ayuric® is a standardized aqueous extract of the fruits of the Terminaha bellerica plant, brown color powder and soluble in water with astringent taste. It contains not less than 15% w/w of low molecular weight hydrolysable tannins as bioactives, including chebulinic acid and chebulagic acid and other unidentified low molecular weight hydrolysable tannins. Ellagic acid and gallic acid are also present in the extract, for which the analytical results may be reported without any specification, but specifications for these bioactives may also be identified. In an embodiment, Ayuric® is a composition of this invention and contains 33-38% w/w low molecular weight hydrolysable tannins, including chebulinic acid and chebulagic acid and other unidentified low molecular weight hydrolysable tannins, and in a further embodiment, also contains 0.1-8% w/w gallic acid and 0.1-8% w/w ellagic acid. In the below Example, Ayuric® had the appearance of a powder with occasional lumps, off-white to brown in color, with an astringent taste, 31.53% w/w total low molecular weight hydrolysable tannins (chebulainic acid and chebulagic acid+other low molecular weight hydrolysable tannins), 4.95% w/w gallic acid; 0.48% w/w ellagic acid; moisture content of 4.36% w/w (meeting standardized requirement of 6% or less), and 94.29% water-soluble extractive value (by gravimetry, meeting standardized requirement of 80% w/w or more). In an embodiment, a composition of this invention may be a preparation of Terminalia bellerica fruit, dried and powdered and in an embodiment, standardized for pharmaceutical or nutraceutical usage. In an embodiment, an extract of this invention may be prepared from such a fruit preparation.

Phyllanthus emblica (Capros®)

In the present invention, an extract is prepared from P. emblica fruit by disrupting the fruit from its natural state and treating the fruit with water or aqueous solution such as phosphate buffered saline (PBS) or other aqueous solution with for instance a salt, pH, and/or other chemical component(s) to form the aqueous extract. In keeping other disclosures herein, in the present invention, a “standardized aqueous extract” is an extract in which specific components have been identified and present in a minimum or maximum amount or a specific range, so as to render the extract consistent at least with regard to those components from one batch to the next. In an embodiment an extract, preferably a standardized aqueous extract, of P. emblica fruit is 60% (w/w) or greater low molecular weight hydrolyzable tannins, for instance 70% (w/w) or greater, or 80% (w/w) or greater; and/or about 5% (w/w) or less gallic acid, for instance 4% (w/w) or less, or 3% (w/w) or less, or 2% (w/w) or less gallic acid. In an embodiment, low molecular weight according to the present invention refers to a molecular weight of less than 1000 daltons.

In an embodiment, a standardized aqueous extract of this invention is prepared by extracting finely pulped P. emblica fruit with a dilute aqueous or alcoholic-water salt solution, for instance 0.1-5% (w/w) sodium chloride solution or 0.1-5% (w/w) sodium citrate/citric acid, or the like, such as 1% NaCl (w/w), preferably at a temperature of about 70° C. (e.g. 65-75° C.) to form an extract-containing solution, filtering the solution, and drying to provide the extract as a powder. In an embodiment, one or more processes for preparing a P. emblica extract of the present invention is described in U.S. Pat. No. 6,124,268, and is incorporated by reference herein to describe said process(es).

In an embodiment, a standardized aqueous extract of P. emblica fruits according to this invention is Capros® (Natreon, New Brunswick, N.J.). Capros® is a preferred extract of P. emblica fruit of this invention, and is the P. emblica extract used in the below Example. Capros® is a super antioxidant, is all natural, completely water soluble and stable, suitable for both solid dosage forms as well as powdered forms, for instance for hot and cold beverages. In an embodiment, Capros® has the appearance of a yellow free-flowing powder, with an astringent taste. The powder has a water-soluble extractive value of greater than or equal to 80% (w/w). In an embodiment, said value is greater than 90%, or greater than 95%. In an embodiment, Capros® powder includes greater than or equal to 60% (w/w) low molecular weight hydrolysable tannins, including for instance greater than 70% or greater than 75%; has a gallic acid content less than or equal to 4% (w/w), including for instance less than 2%, or less than 1%; has a water content of less than or equal to 6% (w/w), including less than 5% w/w, less than 4% w/w, less than 3% w/w, or less than 2% w/w; and has a sulfated ash content of less than or equal to 6% (w/w). In an embodiment, Capros® includes fewer than 10 ppm heavy metals, for instance less than 2 ppm; 5000 CFU/g aerobic bacteria or less (including for instance less than 1000 CFU/g or less than 20 CFU/g); and no measurable Escherichica coli or Candida albicans in 1 g powder, or Salmonella species, Pseudomonas aeruginosa, and/or Staphylococcus aureus in 10 g powder. In the below Example, Capros® had the appearance of a fine powder with occasional lumps, cream to pale yellow color, astringent taste, 71.36% w/w low molecular weight hydrolysable tannins, 0.17% w/w gallic acid, mucic acid-2-0 gallate 16.69% w/w, mucic acid-1,4-lactone-5-O-gallate 4.11% w/w, galloyl glucose 16.58% w/w, with 100% of particles passing through 40 and 80 mesh sizes, bulk density 0.56 g/cc, moisture content 3.88% w/w, sulfated ash 5.02% w/w, and water-soluble extractive value (by gravimetry) 88.01%.

In an embodiment, Capros® is prepared by washing and de-pulping the fresh P. emblica fruits, pressing and centrifuging the pulp to squeeze the juice out, mixing the juice with small percentages of sodium chloride to prevent oxidative decomposition, sodium benzoate or a natural preservative to prevent bacterial growth, and optionally 10-30% maltodextrin as a carrier and silicon dioxide as an anti-caking and anti-sticking agent. The mixture is then spray-dried into a powder and stored.

In an embodiment, a standardized aqueous extract of this invention is in powdered form and may be blended together with other substances in powdered form. In another embodiment, the aqueous standardized extract may be in liquid form, for instance as prepared or for instance as a powder dissolved into water or other liquid. A composition of the present invention may further comprise one or more excipients, additives, and/or other substances, including for instance microcrystalline cellulose, croscarmellose sodium, magnesium stearate, and/or silicon dioxide; and/or a suitable aqueous solution such as a buffer solution. A composition of the present invention may be formulated into nutraceutical or pharmaceutical dosage forms comprising for instance tablets, capsules, powders, liquids, chews, gummies, lozenges, pills, and so forth. In an embodiment, a composition of the present invention is the composition administered as in the Example below, and/or used to prepare the composition.

Azadirachta indica (PhytoBGS®)

PhytoBGS® of this invention is a standardized aqueous extract of the leaves and twigs of Azadirachta indica plant, brown color powder and soluble in water with bitter taste. It contains not less than 2% w/w of flavonoids (comprising of quercetin-3-O-glucoside, quercetin-3-O-rutinoside, apigenin rutionoside and other rutin derivatives) and not less than 5% w/w and up to 20% w/w of myoinositol monophosphate as bioactives, and is devoid of possibly toxic compounds such as azadirachtone, azadiradione, nimbolide, nimbin. In an embodiment, a PhytoBGS® composition of this invention contains about 3% w/w flavonoids and 6-8% w/w myoinositol monophosphate. In the below Example, the PhytoBGS® had the appearance of a fine free-flowing powder, brownish in color, bitter taste, 88.30% water soluble extractive value, by HPLC 3.72% w/w total flavonoids (including quercetin-3-O-glucoside, quercetin-3-O-rutinoside, apigenin rutinoside, and rutin derivatives), 8.69% w/w myo-inositol monophosphate, 3.62% w/w water content (Karl Fischer), 6.52% w/w total polyphenols, 12.90% w/w total amino acids (including arginine and methionine), 0.35 g/cc bulk density, 11.98% w/w total ash, particle size 100% w/w passing through mesh #40 and 99% w/w passing through mesh #80.

### Crominex®-3+

Trivalent chromium complex (for instance, Crominex-3+®) Crominex-3+® of this invention is a complex of trivalent chromium chloride, Phyllanthus emblica extract and Shilajit extract. It is a light brown color powder, soluble in water with astringent taste. It contains not less than 2% w/w of trivalent chromium as the bioactive. In an embodiment, a composition of this invention is Crominex-3+® containing 2-3% of trivalent chromium. In the below Example, Crominex®-3+ had the appearance of a free-flowing powder, greenish brown color, 229.8 ug Chromium III in 10 mg with Chromium⁶⁺ absent (required standard: 200 ug or more in 10 mg, with Chromium⁶⁺ absent), Chromium

### 3,8-dihydroxy-6H-benzo[c]chromen-6-one and 3-hydroxy-6H-benzo[c]chromen-6-one

3,8-dihydroxy-6H-benzo[c]chromen-6-one, in an embodiment, is a pale brown powder (typically a pale yellow to yellowish green powder or pale brown to dark brown powder), soluble in DMSO and largely insoluble in water. IR spectra of a reference standard may be used to confirm identify. 3-hydroxy-6H-benzo[c]chromen-6-one, in an embodiment, is an off-white to pinkish brown color powder, soluble in DMSO and DMF (dimethylformamide) but largely insoluble in water. IR spectra of a reference standard may be used to confirm identify. Other extracts of the present invention that may be included in a composition of this invention include for instance extracts prepared as described in Table 2 below.

TABLE 2
Hydroalcoholic systems and extracts of
Withania somnifera (Ashwagandha)

| Product | Solvent used for Extraction |
|---|---|
| Ashwagandha | 100% ethanol extraction followed by water |
| Leaf Extract | extraction. This process allows extraction of |
| Ashwagandha | alcohol-soluble as well as water-soluble |
| Root Extract | bioactives. Here, alcohol and water are |
| Ashwagandha | used separately. |
| Root & Leaf | |
| Extract | |
| Ashwagandha | The solvent used in these batches is a |
| Root & Leaf | hydro-alcoholic system with ethanol:water: |
| Ashwagandha | 70:30 (v/v). This solvent system also allows |
| Root Extract | extraction of alcohol-soluble as well as |
| Ashwagandha | water-soluble bioactives. Here, alcohol and |
| Leaf Extract | water are used in combination. |

The above extracts, extract components, extract systems, extract preparation procedures, and other related information, and also extraction and other preparation methodologies described throughout the application, are intended as exemplary and not as limiting in the context of this invention.

### Additional Extraction and Other Preparation Methodologies

In an embodiment, a method of manufacturing of a T. chebula fruit extract is described in U.S. Pat. No. 10,500,240, which is incorporated by reference herein for this purpose. The same method may be used for manufacturing T. bellerica extract also.

In an embodiment, the extraction process of the current invention includes the steps of providing dried fruits of T. chebula or T. bellerica, de-seeding the fruits, pulverizing or grinding the pulp to a powder, extracting the pulp powder with an extraction solvent or solvent mixture, optionally, with heating, to provide a T. chebula or T. bellerica enriched liquid extract, optionally concentrating the liquid extract and drying the concentrated liquid extract to provide a hydrolyzable tannoid enriched T. chebula or T. bellerica extract powder. Aqueous solvent is preferred. A particularly preferred solvent is water. Useful extraction temperatures can range from about 25° C. (ambient) to about 90° C. Particularly useful extraction temperatures can range from about 25° C. to about 80° C.

In an embodiment, AyuFlex® and Ayuric® are prepared in keeping with the methods described above.

In another embodiment, ethanol or methanol or a hydro-alcoholic mixture may be used as the solvent system for extraction.

In an embodiment, useful extraction times in conjunction with maintaining useful temperatures can range from about 2 hours to about 16 hours. A particularly useful extraction time range at about 25°±5° C. is from about 12 hours to about 16 hours, and at a temperature of 40°±5° C. is from about 2 hours to about 6 hours. Length and temperature of extraction may be varied at atmospheric pressure (i.e., approx. 1 atm). It is contemplated that pressure can be varied in the extraction process, for example, by use of a commercial pressure reactor apparatus.

The extraction process can also include drying the liquid extract to a powder form. Suitable drying methods include spray drying, lyophilization (freeze drying), vacuum drying (with or without heating), evaporation (with or without heating), and concentration under vacuum. Once isolated or obtained, the hydrolyzable tannoid enriched T. chebula or T. bellerica extract powder may be processed by any suitable means, including grinding, milling, sieving, sizing, blending and the like. The obtained hydrolyzable tannoid enriched T. chebula or T. bellerica extract powder may be prepared in any suitable particle size or particle size range.

Process additives such as microcrystalline cellulose, starch, maltodextrin and the like as carrier materials, anti-adherents such as silicone dioxide, rice bran powder and the like, and preservatives such as sodium benzoate, methyl paraben, propyl paraben, natural preservatives and the like may be added during the extraction process or during the final blending of the dried extract powder. In an embodiment, such may be used in compositions having other extracts of this invention as well.

In the case of Phyllanthus emblica extract, seeds are removed from the fresh fruits after washing, the juice is separated from the pulp by pressing and/or centrifugation and the juice is dried by spray drying or another suitable drying technique, such as microwave drying, freeze drying, etc. Additives, such as preservatives, such as sodium chloride and sodium benzoate, carrier materials such as maltodextrin, microcrystalline cellulose, anti-adherent such as silicon dioxide and rice bran powder may also be added, optionally.

In the cases of Withania somnifera, Shilajit and Azadirachta indica, the dried roots and/or leaves of the plant, the shilajit stone, and the dried leaves and twigs respectively, are milled and subjected to extraction and drying. Extraction is done using water only as the solvent or an alcohol, preferably ethanol or methanol, followed by water. The liquid extract is then optionally concentrated by evaporation and then dried by spray drying, freeze drying, microwave drying or another suitable drying technique. Extraction temperatures may range from 40° C.±5° C. to 90° C.±5° C., preferably 60° C.±5° C. to 80° C.±5° C. Extraction times may vary from 1 hour to 12 hours, preferably from 2 hours to 6 hours. Several cycles of extraction may also be done. In an embodiment of Azadirachta indica, only leaves may be used as the starting raw material.

Trivalent chromium complex is made by dissolving trivalent chromium chloride in water, with or without heat, adding Phyllanthus emblica extract and mixing for a suitable length of time to form a complex, then adding shilajit and mixing for a suitable length of time, then adding a carrier material such as microcrystalline cellulose, maltodextrin, starch, etc. and mixing for a suitable length of time, followed by spray drying, freeze drying, microwave drying or another suitable drying method. The temperature of the complexation step may be from 30° C.±5° C. to 80° C.±5° C., preferably from 40° C.±5° C. to 60° C.±5° C.

A composition of the present invention may be formulated into nutraceutical or pharmaceutical dosage forms comprising for instance tablets, capsules, powders, liquids, chews, gummies, transdermals, injectables, dietary supplements, topical creams, lozenges, pills, and so forth. A composition of the present invention may further comprise one or more excipients, additives, and/or other substances, including for instance microcrystalline cellulose, croscarmellose sodium, magnesium stearate, and/or silicon dioxide. A powdered blend of one or more extracts and optionally excipients or other substances such as fillers, disintegrants, flow enhancers, lubricants, and/or other substances discussed throughout this application, including for instance, microcrystalline cellulose, croscarmellose sodium, silicon dioxide, and magnesium stearate, may be blended using standard powder blending techniques. In an embodiment, a composition of this invention may be marked as organic by an appropriate agency or organization. In an embodiment, a composition of the present invention in a dosage form includes for instance from 1-2000 mg of an extract and/or extract component of Table 1 or Table 2.

In the present invention, an “effective amount” refers to an amount of an extract and/or extract component of this invention, comprised in a composition of this invention, which is administered to a subject and acts at the subject's respiratory system and/or bloodstream and/or bodily cells and/or tissues to protect cells from viral infection and to thereby treat and/or prevent viral infection in the subject, and accordingly its symptoms and deleterious effects; in an embodiment to treat, prevent, and/or protect from coronavirus infection such as 229E or SARS-CoV-2 infection (COVID-19). In an embodiment, an “effective amount” refers to an amount of extract and/or extract component administered directly to cells. In an embodiment, an effective amount of an extract of this invention is administered to different or additional system(s), organ(s), tissue(s), and/or cell(s) to protect the subject from viral infection.

In an embodiment, an effective amount of extract and/or extract component of a composition of this invention is a daily dose of 1 mg-2000 mg extract and/or extract component to a subject, as discussed below and throughout this application. In an embodiment, administration is to a human subject; in an embodiment, as a dietary supplement. In an embodiment, an effective amount of extract and/or extract component is a daily dose of about 100-1000 mg, for instance administered as a dose of about 50 mg to 500 mg, twice daily (or once daily). In an embodiment, a composition comprising one or more standardized extracts of this invention is administered in an effective amount to a mammal, including a daily dose for a human being of at least 1-2000 mg of at least one extract, in an embodiment at least 50 mg, 100 mg, 250 mg, 500 mg, 750 mg, 1000 mg, 1500 mg, or 2000 mg; and optionally 1-2000 mg of each and any other extract included in the composition. In a mammal, a dose may be about the same as in a human, and may be adjusted per kilogram of weight of the mammal. In an embodiment, PrimaVie® is administered to a subject, in an embodiment a human subject, in a dose of about 100 mg to about 1000 mg daily, for instance in doses of about 50 mg to about 500 mg twice daily. In an embodiment, Urolithin A and/or Urolithin B are administered to a subject, in an embodiment a human subject, in a dose of about 100 mg to about 1000 mg daily, for instance in doses of about 50 mg to about 500 mg twice daily. In an embodiment, PrimaVie® and Urolithin A and/or Urolithin B are administered to a subject, in an embodiment a human subject, in a dose of about 1 mg to about 2000 mg, in an embodiment 100 mg to about 1000 mg daily, for instance in doses of about 50 mg to about 500 mg twice daily. In an embodiment, when more than one substance (for instance extract PrimaVie®, extract component Urolithin A, and/or extract component Urolithin B) is administered, the dosage administered may be the total amount of the substances administered (e.g. 100-1000 mg daily total of all of these substances in any combination), or the dosage administered may be the amount of each substance administered (for instance, a composition comprising PrimaVie® and Urolithin A may include 100-1000 mg of PrimaVie® and 100-1000 mg of Urolithin A). Other combinations of extracts and extract components may be used in this infention. In an embodiment, Sensoril® is administered to a subject, in an embodiment a human subject, in a dose of about 100 mg to about 1000 mg daily, for instance in doses of about 50 mg to about 500 mg twice daily.

A “dietary supplement” according to the present invention refers to a composition comprising an extract and/or extract component of this invention which is administered as an addition to a subject's diet, which is not a natural or conventional food, which when administered to the subject reaches and acts at the subject's respiratory system and/or bloodstream and/or bodily cells and/or tissues to protect against viral infection, in an embodiment to protect from coronavirus infection, in an embodiment to protect from SARS-CoV-2 infection. In an embodiment, a dietary supplement containing an effective amount of an extract and/or extract component according to the present invention is administered orally. In an embodiment, the dietary supplement is administered for instance daily or twice daily in an amount of about 1-2000 mg, for instance 100-1000 mg daily, for instance 50-500 mg twice daily; in an embodiment, the dietary supplement is administered for 1-7 days or more, or for another period of time according to the present invention. A dietary supplement may be formulated into various forms, as discussed throughout this application. In an embodiment, a dietary supplement or other composition of this invention is administered to a subject prior to the subject's exposure to a virus such as a coronavirus such as 229E or SARS-CoV-2, to prevent infection of the subject and to protect cells from infection by the virus. In an embodiment, such pre-treatment prior to cells or a subject's exposure to the virus is preferably at least 1 hour prior, 12 hours prior, 24 hours prior, and/or 1-60 days prior to exposure to the virus. In an embodiment, “pre-treatment” with a composition of this invention may be referred to as preventive or as treatment according to this invention.

“Treatment” and the like according to the present invention refers to reducing and/or eliminating cell death from viral infection and/or improving cell viability in the presence of a virus/viral infection by protecting cells from viral infection via administration of a composition of this invention. Without being bound by theory, treatment according to this invention may include reducing or eliminating symptoms of viral infection in a subject, reducing or eliminating the occurrence of cell damage as well as tissue, organ, and system damage in a subject, reducing or eliminating serious illness in a subject, including for instance subjects having an underlying condition that may increase their susceptibility to the viral infection, and may lessen or eliminate symptoms, complications, and overall serious illness in a subject. In particular, treatment according to this invention is for COVID-19 and/or other coronavirus infections. Treatment may refer to protection of cells in a subject that already has a viral infection prior to administration of an extract or extract component composition of this invention. Similarly, “prevention” and the like according to the present invention refers to protecting cells from viral infection including coronavirus infection before the cells are infected. Such protection may occur for instance before exposure of a subject to a virus and before viral infection or for instance may occur during an active viral infection by protecting uninfected cells from infection (without being bound by theory). In an embodiment, treatment may refer to administration of a composition of this invention in a subject having a viral infection and prevention may refer to administration of a composition of this invention in a subject that does not have a viral infection such as a coronavirus infection. In an embodiment, treatment and prevention may occur on a cellular level in a subject that has a viral infection.

The present invention may be further understood in connection with the following Examples and embodiments. The following non-limiting Examples and embodiments described throughout this application are provided to illustrate the invention.

### Example 1

The below Example describes a preliminary screening study. Results are not intended as determinative. Follow-on studies with PrimaVie® Shilajit are described in Example 2.

### Methodology

Products Screened: The following products from Natreon, Inc. (New Brunswick, N.J.) were included in the preliminary screening study:
1. Aqueous extract (standardized) of the roots Asparagus racemosus (Vytus-AR™)
2. Aqueous extract (standardized) of the roots+aerial parts of Withania somnifera (Sensoril®)
3. Aqueous extract (standardized) of the fruits of Phyllanthus emblica (Capros®)
4. Aqueous extract (standardized) of the fruits of Terminalia chebula (AyuFlex®)
5. Aqueous extract (standardized) of the fruits of Terminalia bellirica (Ayuric®)
6. Aqueous extract (standardized) of the herbo-mineral product, Shilajit (PrimaVie®)
7. Aqueous extract (standardized) of the leaves plus twigs of Azadirachta indica (PhytoBGS®), and
8. A complex of trivalent chromium and the standardized aqueous extracts of Phyllanthus emblica and Shilajit (Crominex®-3+).

### Screening Procedure:

Human lung fibroblast cell line MRC-5 (ATCC® CCL-171™) was seeded at 5000/well in 96 well plates in Eagle's Minimum Essential Medium (EMEM) (ATCC® 30-2003™) supplemented with 10% fetal bovine serum (FBS) and 1% antibiotic-antimycotic. Seeded plates were incubated at 37° C. in a CO2 incubator for 6 hours before pre-treatment with two doses of each of the eight compounds as follows:
Vytus-AR®, Ayuflex®, Ayuric®, Capros®, Crominex-3+® (0.10 mg/ml and 0.25 mg/ml)
PhytoBGS®, PrimaVie®, Sensoril® (0.25 mg/ml and 0.50 mg/ml)

All treatments were prepared by dissolving respective compounds in EMEM and filter sterilizing through a 0.22 μm filter. Each pre-treatment was given for 12 h (n=10) and 24 h (n=10). The treatment medium was removed thereafter from six wells in each Group and infected with 10⁶ human coronavirus 229E (ATCC® VR-740™) in EMEM supplemented with 2% FBS. MRC-5 cells treated with vehicle (EMEM only) and MRC-5 cells infected with 10⁶ 229 E were maintained as controls and all experimental Groups were monitored from day 3 and concluded on day 7 post infection.

### Results:

Pre-treatment with 0.25 and 0.50 mg/ml PrimaVie for 24 hours was found to protect MRC-5 cells against 229E induced cytopathy in 50% of experimental replicates (n=3). Screening results are presented in Tables 3-6.

TABLE 3
12 hours pre-treatment with extract

| | | Treatment | | | | Treatment + 10⁶ 229E | | | | | | Controls | |
|---|---|---|---|---|---|---|---|---|---|---|---|---|---|
| Conc | Compound | 1 | 2 | 3 | 4 | 1 | 2 | 3 | 4 | 5 | 6 | CoV | U |
| 0.10 | Vytus-AR ® | + | + | + | + | − | − | − | − | − | − | − | ++ |
| mg/ml | Ayuflex ® | + | + | + | + | − | − | − | − | − | − | − | ++ |
| | Ayuric ® | + | + | + | + | − | − | − | − | − | − | − | ++ |
| | Capros ® | ++ | ++ | ++ | ++ | − | − | − | − | − | − | − | ++ |
| | Crominex ®-3+ | ++ | ++ | ++ | ++ | − | − | − | − | − | − | − | ++ |
| 0.25 | PhytoBGS ® | ++ | ++ | ++ | ++ | − | − | − | − | − | − | − | ++ |
| mg/ml | PrimaVie ® | ++ | ++ | ++ | ++ | − | − | − | − | − | − | − | ++ |
| | Sensoril ® | ++ | ++ | ++ | ++ | − | − | − | − | − | − | − | ++ |

TABLE 4
12 hours pre-treatment with extract

| | | Treatment | | | | Treatment + 10⁶ 229E | | | | | | Controls | |
|---|---|---|---|---|---|---|---|---|---|---|---|---|---|
| Conc. | Compound | 1 | 2 | 3 | 4 | 1 | 2 | 3 | 4 | 5 | 6 | CoV | U |
| 0.25 | Vytus-AR ® | + | + | + | + | − | − | − | − | − | − | − | ++ |
| mg/ml | Ayuflex ® | | | | | | | | | | | | |
| | Ayuric ® | + | + | + | + | − | − | − | − | − | − | − | ++ |
| | Capros ® | | | | | | | | | | | | |
| | Crominex ®-3+ | ++ | ++ | ++ | ++ | − | − | − | − | − | − | − | ++ |
| 0.50 | PhytoBGS ® | ++ | ++ | ++ | ++ | − | − | − | − | − | − | − | ++ |
| mg/ml | PrimaVie ® | + | + | + | + | − | − | − | − | − | − | − | ++ |
| | Sensoril ® | − | − | − | − | − | − | − | − | − | − | − | − |

TABLE 5
24 hours pre-treatment with extract

| | | Treatment | | | | Treatment + 10⁶ 229E | | | | | | Controls | |
|---|---|---|---|---|---|---|---|---|---|---|---|---|---|
| Conc. | Compound | 1 | 2 | 3 | 4 | 1 | 2 | 3 | 4 | 5 | 6 | CoV | U |
| 0.10 | Vytus-AR ® | ++ | ++ | ++ | ++ | − | − | − | − | − | − | − | ++ |
| mg/ml | Ayuflex ® | + | + | + | + | − | − | − | − | − | − | − | ++ |
| | Ayuric ® | + | + | + | + | − | − | − | − | − | − | − | ++ |
| | Capros ® | ++ | ++ | ++ | ++ | − | − | − | − | − | − | − | ++ |
| | Crominex ®-3+ | ++ | ++ | ++ | ++ | − | − | − | − | − | − | − | ++ |
| 0.25 | PhytoBGS ® | ++ | ++ | ++ | ++ | − | − | − | − | − | − | − | ++ |
| mg/ml | PrimaVie ® | ++ | ++ | ++ | ++ | ++ | ++ | − | − | ++ | − | − | ++ |
| | Sensoril ® | ++ | ++ | ++ | ++ | − | − | ++ | ++ | − | ++ | − | ++ |

TABLE 6
24 hours pre-treatment with extract

| | | Treatment | | | | Treatment + 10⁶ 229E | | | | | | Controls | |
|---|---|---|---|---|---|---|---|---|---|---|---|---|---|
| Conc. | Compound | 1 | 2 | 3 | 4 | 1 | 2 | 3 | 4 | 5 | 6 | CoV | U |
| 0.25 | Vytus-AR ® | − | − | − | − | − | − | − | − | − | − | − | ++ |
| mg/ml | Ayuflex ® | − | − | − | − | − | − | − | − | − | − | − | ++ |
| | Ayuric ® | − | − | − | − | − | − | − | − | − | − | − | ++ |
| | Capros ® | − | − | − | − | − | − | − | − | − | − | − | ++ |
| | Crominex ®-3+ | ++ | ++ | ++ | ++ | − | − | − | − | − | − | − | ++ |
| 0.50 | PhytoBGS ® | ++ | ++ | ++ | ++ | − | − | − | − | − | − | − | ++ |
| mg/ml | PrimaVie ® | ++ | ++ | ++ | ++ | − | ++ | ++ | − | − | ++ | − | ++ |
| | Sensoril ® | − | − | − | − | − | − | − | − | − | − | − | ++ |

Key: ++ implies 100% confluent cells; + implies 50-70% confluent cells; − implies cytopathic effects

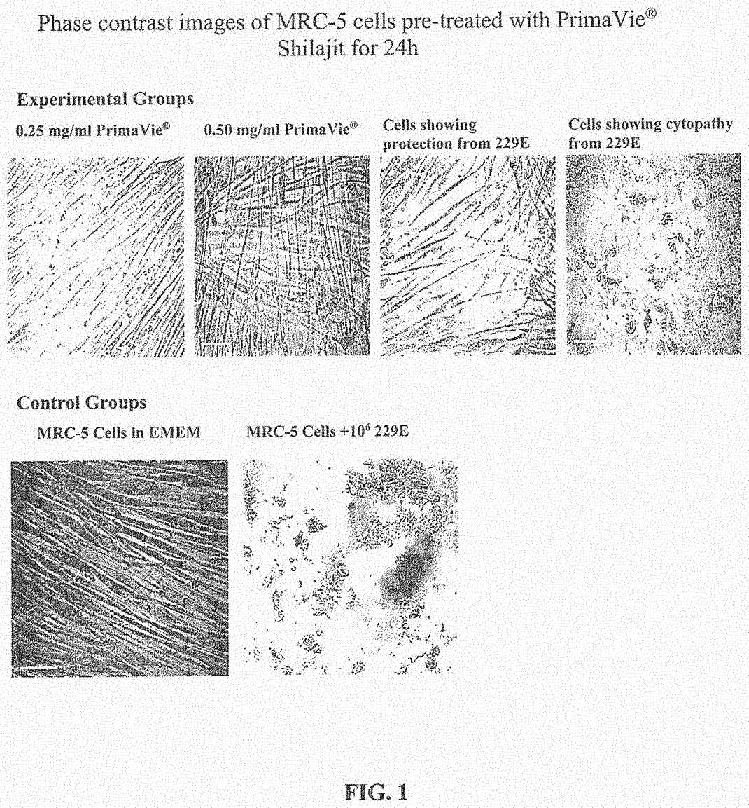

FIG. 1 shows phase contrast images of MRC-5 cells pre-treated with 0.25 mg/ml and 0.50 mg/ml of Shilajit PrimaVie® for 24 h, and exposed to coronavirus 229E. Photomicrographs are presented in black and white, with elongate cells presenting as viable and small round circular structures indicating cell death. The images show elongated cells protected from 229E by PrimaVie® Shilajit, and substantial cell death in cells infected by 229E but not protected by PrimaVie®. See Control Group MRC-5 Cells in EMEM (bottom left) and Experimental Groups 0.25 and 0.5 mg/ml PrimaVie® (1^{st} and 2d photomicrographs, top left), showing normal elongated cells not exposed to coronavirus. Cells exposed to coronavirus 229E and to PrimaVie® show protection from 229E in the top row, third photomicrograph from left, and look similar to the normal cells. Cells showing cytopathy from coronavirus 229E infection are shown at top right of the Experimental Groups and also to the right in the Controls. Images in the 1^{st} row of FIG. 1 are of the same magnification, and images in the 2d row of FIG. 1 are of the same magnification.

Example 2 was conducted with PrimaVie® Shilajit as follows:

### Example 2

Effect of PrimaVie® on Coronavirus Infection of Human Lung Fibroblasts (MRC-5)

### Experimental Procedure

1. Human lung fibroblasts (MRC-5, 1.4×10⁶ total cells) were seeded in 3-T75 flasks and pre-treated with PrimaVie® (0, 0.25 and 0.5 mg/ml) for 72 h (3d).
2. After 72 h (3d), cells were trypsinized from each Group and were seeded @ 5000 cells per well in 96 well plate in 200 μl media.
3. 12 h post-seeding, cells were infected with 10⁶ 229 E (CoV) with/without respective co-treatments {PrimaVie® (0, 0.25 and 0.5 mg/ml)}.
4. 120 h (5d) post-seeding, cell death and viability were analyzed using Calcein-AM-PI staining and MTT assay respectively.

Calcein AM is a cell-permeant dye that can be used to determine cell viability in most eukaryotic cells. In live cells the nonfluorescent Calcein AM is converted to a green-fluorescent calcein after acetoxymethyl ester hydrolysis by intracellular esterases, while propidium iodide is cell-impermeant when the cell wall is intact, but in case of dead cells, the cell wall being breached, it gets inside the cells and reacts with DNA showing as red dots. Thus, the green cells are viable elongate cells and red dots represent dead cells. As FIG. 2 is represented in black and white, the green and red colors are not shown, but the viable elongate cells and round dots showing cell death are evident.

The MTT assay is a colorimetric assay for assessing cell metabolic activity. NAD(P)H-dependent cellular oxidoreductase enzymes may, under defined conditions, reflect the number of viable cells present. These enzymes are capable of reducing the tetrazolium dye MTT dimethylthiazol-2-yl)-2,5-diphenyltetrazolium bromide to its insoluble formazan, which has a purple color, which can be quantified by a colorimetric assay.

### Results:

FIG. 2 shows photomicrographs showing human lung fibroblast (MRC-5) cell death caused by coronavirus infection (229E) and protection by PrimaVie® using Calcein-AM and Propidium Iodide Staining. Viable cells are elongate, and dead cells present as small and round. The top row shows normal, elongate MRC-5 cells with or without PrimaVie® treatment. The bottom row shows MRC-5 cells infected with 229E coronavirus as discussed above, with or without PrimaVie® treatment. As may be seen from the far right photomicrographs, PrimaVie® at 0.5 mg/ml concentration provided full protection to the human lung fibroblasts from coronavirus infection, while at 0.25 mg/ml, there appears to be a partial protection.

FIG. 3 shows a bar graph showing human lung fibroblast (MRC-5) cell death caused by coronavirus infection (229E) and protection by PrimaVie® using Calcein-AM and Propidium Iodide Staining.

FIG. 4 shows a bar graph showing human lung fibroblast (MRC-5) cell viability by coronavirus infection (229E) and protection by PrimaVie® using MTT assay.

It can be seen from both Calcein-AM and Propidium Iodide staining as well as the MTT assay that PrimaVie® Shilajit extract indeed protects the human lung fibroblast cells from coronavirus infection. PrimaVie® Shilajit extract has dibenzo-alpha-pyrones (Urolithins A and B), fulvic acid and humic acids as bioactive constituents.

### Example 3

Additional experimentation with PrimaVie® Shilajit is expected to treat, prevent, and/or protect subjects and cells from viral infection such as from coronavirus and COVID-19.

### Example 4

As shown in Example 1, an aqueous extract of Ashwagandha (Withania somnifera), including the entire plant, offers protection to human lung fibroblasts. Additional experimentation with Withania somnifera extracts, such as the hydroalcoholic extracts described in Table 2, is expected to show the protection of cells and of subjects from viral infection.

The use of the terms “a,” “an,” “the,” and similar referents in the context of describing the present invention (especially in the context of the claims) are to be construed to cover both the singular and the plural, unless expressly otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied. All method steps described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non-claimed element as essential to the practice of the invention.

While in the foregoing specification the present invention has been described in relation to certain embodiments thereof, and many details have been put forth for the purposes of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

1. A method of treating and/or preventing viral infection in a subject comprising the steps of:
a. providing a composition comprising an extract and/or an extract component, and
b. administering an effective amount of the composition to the subject to treat and/or prevent the viral infection.

2. The method of claim 1, wherein said viral infection is by a coronavirus.

3. The method of claim 2, wherein said coronavirus is SARS-CoV-2 or 229E.

4. The method of claim 2, wherein said method is for the treatment and/or prevention of COVID-19.

5. The method of claim 1, wherein said extract is a standardized aqueous extract of Shilajit.

6. The method of claim 1, wherein said extract is a standardized extract of Withania somnifera.

7. The method of claim 1, wherein said extract component is 3,8-dihydroxy-6H-benzo[c]chromen-6-one and/or 3-hydroxy-6H-benzo[c]chromen-6-one.

8. The method of claim 5, wherein the effective amount of said composition comprises a daily dose of 100-1000 mg standardized aqueous extract of Shilajit in a human subject.

9. The method of claim 6, wherein the effective amount of said composition comprises a daily dose of 100-1000 mg standardized extract of Withania somnifera in a human subject.

10. The method of claim 7, wherein the effective amount of said composition comprises a daily dose of 100-1000 mg 3,8-dihydroxy-6H-benzo[c]chromen-6-one and/or 3-hydroxy-6H-benzo[c]chromen-6-one combined.

11. The method of claim 1, wherein the effective amount is administered to the subject at least 24 hours before exposure to a virus.

12. The method of claim 1, wherein the effective amount of said composition comprises 50-500 mg of said extract or extract component, administered to a human subject twice daily, for 1-7 consecutive days.

13. The method of claim 1, wherein said treatment and/or prevention reduces or eliminates cell death in the subject from the viral infection.

14. The method of claim 1, wherein said treatment and/or prevention reduces or eliminates symptoms of the viral infection in the subject.

15. The method of claim 1, wherein said treatment and/or prevention reduces or eliminates cellular, tissue, and/or organ damage to the subject from the viral infection.

16. The method of claim 1, wherein said treatment and/or prevention reduces the risk of serious illness or reduces serious illness from the viral infection in the subject.

17. The method of claim 1, wherein said treatment and/or prevention reduces the risk of serious illness from the viral infection in the subject wherein the subject has underlying conditions.

18. The method of claim 1, wherein said prevention further comprises administering the composition to the subject, wherein the subject does not have a viral infection, to protect the subject from a future viral infection.
